(19) United States
(12) Patent Application Publication
(10) Pub. No.: US 2023/0157903 A1
(43) Pub. Date: May 25, 2023

(54) SENSOR, DETECTION SYSTEM, AND DETECTION METHOD

(71) Applicant: ABLIC Inc., Tokyo (JP)

(72) Inventors: Fumiyasu UTSUNOMIYA, Tokyo (JP); Ami TANAKA, Shiga (JP); Takakuni DOUSEKI, Shiga (JP); Shiyuya IMOTO, Shiga (JP)

(73) Assignee: ABLIC Inc., Tokyo (JP)

(21) Appl. No.: 17/958,421

(22) Filed: Oct. 2, 2022

(30) Foreign Application Priority Data
Nov. 24, 2021 (JP) 2021-190475

(51) Int. Cl. A61F 13/42; G01N 27/12

(52) U.S. Cl. CPC A61F 13/42; G01N 27/121; A61F 2013/424

(57) ABSTRACT

Provided is a sensor having different sensitivities depending on the positions. A sensor 1 is a sensor for detecting the presence of a liquid, and includes a first electrode 11 and a second electrode 12. The first electrode and the second electrode each have a thread-like or band-like structure, and are arranged side by side in a direction intersecting a longitudinal direction. At least one of the first electrode and the second electrode includes a first portion 111 having a first surface area at a first position in the longitudinal direction, and includes a second portion 112 having a second surface area larger than the first surface area at a second position in the longitudinal direction different from the first position.

### CROSS-REFERENCE TO RELATED APPLICATION

This application claims the priority benefits of Japanese application no. 2021-190475, filed on Nov. 24, 2021. The entirety of the above-mentioned patent application is hereby incorporated by reference herein and made a part of this specification.

### BACKGROUND

### Technical Field

The present invention relates to a sensor, a detection system, and a detection method.

### Description of Related Art

Patent Literature 1 (Japanese Patent Laid-Open No. 2019-166040) discloses that the size of the non-contact range with a liquid in an electrode in an absorbing member is detected by utilizing the amount of parasitic resistance which changes according to the size of the non-contact range with the liquid in the electrode.

### SUMMARY

The inventors have found that it is effective to vary the sensitivity of a sensor depending on the positions, for example, when the sensor is used on diapers or the like which have a position for intensively detecting the presence of a liquid and a position for preliminarily detecting the presence of a liquid. One of the objects of the present disclosure is to provide a sensor having different sensitivities depending on the positions, a detection system using the sensor, and a detection method.

In accordance with an embodiment of the present invention, a sensor is provided for detecting presence of a liquid, and the sensor includes a first electrode and a second electrode. The first electrode and the second electrode each have a thread-like or band-like structure, and are arranged side by side in a direction intersecting a longitudinal direction for detecting the liquid existing between the electrodes. At least one of the first electrode and the second electrode includes a first portion having a first surface area at a first position in the longitudinal direction, and includes a second portion having a second surface area larger than the first surface area at a second position in the longitudinal direction different from the first position.

In accordance with an embodiment of the present invention, a detection system is provided for detecting presence of a liquid in an object to be detected, and the detection system includes a sensor having a first electrode and a second electrode and mounted on the object to be detected; a communication device connected to the sensor and outputting a sensing result of the sensor by wireless communication; and a receiver receiving the sensing result. The first electrode and the second electrode each have a thread-like or band-like structure, and are arranged side by side in a direction intersecting a longitudinal direction. At least one of the first electrode and the second electrode includes a first portion having a first surface area at a first position in the longitudinal direction, and includes a second portion having a second surface area larger than the first surface area at a second position in the longitudinal direction different from the first position.

In accordance with an embodiment of the present invention, a detection method is provided for detecting presence of a liquid. A first electrode and a second electrode each having a thread-like or band-like structure are arranged side by side in a direction intersecting a longitudinal direction. The first electrode and the second electrode are made of different materials, and generated power is generated between the electrodes when the liquid is present between the electrodes as the electrodes come into contact with the liquid existing between the electrodes. At least one of the first electrode and the second electrode includes a first portion having a first surface area at a first position in a longitudinal direction, and includes a second portion having a second surface area larger than the first surface area at a second position in the longitudinal direction different from the first position. The fact that a predetermined amount of the liquid is present is detected by detecting that an amount of the generated power exceeds a threshold value. The threshold value is a value larger than a maximum value of an amount of generated power of the first portion and smaller than a maximum value of an amount of generated power of the second portion.

Further details will be described as embodiments provided below.

### BRIEF DESCRIPTION OF THE DRAWINGS

FIG. 1 is a schematic view of the detection system 100 according to an embodiment.

FIG. 2 is a cross-sectional view of the absorbing member body taken along the line M-M of FIG. 1.

FIG. 3 is a schematic view illustrating the configuration of the detection device.

FIG. 4 is a diagram illustrating the measurement results of the generated current of the sensor obtained by the inventors.

FIG. 5 is a diagram illustrating the measurement results of the change over time of the charge voltage of the capacitor obtained by the inventors.

FIG. 6 is a diagram illustrating the measurement results of the radio signal, obtained in an experiment of detecting the replacement time of the absorbing member body by the detection system, obtained by the inventors.

### DESCRIPTION OF THE EMBODIMENTS

<1. Overview of the Sensor, the Detection System, and the Detection Method>

(1) The sensor according to an embodiment is a sensor for detecting the presence of a liquid, and includes a first electrode and a second electrode. The first electrode and the second electrode each have a thread-like or band-like structure, and are arranged side by side in a direction intersecting a longitudinal direction. At least one of the first electrode and the second electrode includes a first portion having a first surface area at a first position in the longitudinal direction, and includes a second portion having a second surface area larger than the first surface area at a second position in the longitudinal direction different from the first position.

By setting the second surface area of the second portion to be larger than the first surface area of the first portion, the second portion has a larger area in contact with the liquid than the first portion. Therefore, when the same amount of liquid is present between the first portion and the second portion between the electrodes, the detection sensitivity of the presence of the liquid between the electrodes of the second portion is improved, compared to the detection sensitivity between the electrodes of the first portion. That is, the sensitivity can be made different depending on the positions in the longitudinal direction of the sensor.

(2) According to an embodiment, the first electrode and the second electrode are made of different materials, and generated power is generated between electrodes in response to the liquid being present between the electrodes, and second generated power generated by the second portion is larger than first generated power generated by the first portion. Since the second generated power is larger than the first generated power, the sensitivity of the sensor which detects the presence of the liquid by using the generated power can be made different in the longitudinal direction.

(3) According to an embodiment, the sensor further includes an output part which outputs a detection signal indicating detection of the liquid, and the output part is connected to the first electrode and the second electrode. Thus, the sensor can output a detection signal according to the generated power.

(4) According to an embodiment, the first electrode and the second electrode are provided so as to be in contact with an absorbent of a worn article having the absorbent which receives excrement, and the first position is located in front of the second position in the case of a wearer wears the worn article. The worn article having the absorbent which receives excrement is, for example, a diaper.

By providing the first electrode and the second electrode to be in contact with the absorbent of the worn article having the absorbent which receives excrement, the sensor can detect whether there is excrement on the worn article by the liquid contained in the excrement. By providing the first electrode and the second electrode to put the first position in front of the second position when the wearer wears the worn article, the rear sensitivity can be made higher than the front sensitivity. When excrement comes from the front of the worn article and the wearer is in a supine position, the liquid absorbed by the absorbent proceeds rearward, and the entire absorbent absorbs the liquid. By making the rear sensitivity higher than the front sensitivity, it is possible to detect the state where the entire absorbent absorbs the liquid, that is, the timing of replacement in the case of a diaper.

(5) According to an embodiment, at least one of the first electrode and the second electrode is sewn on a fabric. Thus, the surface area can be easily made different between the first portion and the second portion.

(6) According to an embodiment, the first portion and the second portion have different sewing structures of the electrodes with respect to the fabric. The sewing structure refers to a structure which the electrode forms with respect to the fabric as the electrode is sewn on the fabric. Since the electrode has a thread-like or band-like structure, the surface area can be easily made different between the first portion and the second portion by changing the sewing structure.

(7) According to an embodiment, the sewing structure of the second portion has a higher density of electrode with respect to the fabric per unit than the sewing structure of the first portion. Thus, the second surface area which can be in contact with the liquid can be made larger than the first surface area.

(8) According to an embodiment, the first electrode and the second electrode are in contact with each other via a water absorbent, at least one of the first electrode and the second electrode is sewn with the water absorbent as the fabric, and the sewing structure of the second portion has a shorter sewing pitch than the sewing structure of the first portion. Thus, the second surface area which can be in contact with the liquid can be made larger than the first surface area.

(9) According to an embodiment, the sewing structure of the second portion has a larger area of electrode appearing on a surface of the fabric in contact with the liquid than the sewing structure of the first portion. Thus, when the surface of the fabric is in contact with the absorbent, the second surface area which can be in contact with the liquid can be made larger than the first surface area.

(10) According to an embodiment, the sewing structure of the second portion has an overlap with respect to a sewing direction of the electrode. Thus, the second surface area which can be in contact with the liquid can be made larger than the first surface area.

(11) The detection system according to an embodiment is a system for detecting the presence of a liquid in an object to be detected, and includes a sensor having a first electrode and a second electrode and mounted on the object to be detected; a communication device connected to the sensor and outputting a sensing result of the sensor by wireless communication; and a receiver receiving the sensing result. The first electrode and the second electrode each have a thread-like or band-like structure, and are arranged side by side in a direction intersecting a longitudinal direction, and at least one of the first electrode and the second electrode includes a first portion having a first surface area at a first position in the longitudinal direction, and includes a second portion having a second surface area larger than the first surface area at a second position in the longitudinal direction different from the first position.

By making the second surface area of the second portion larger than the first surface area of the first portion, the sensitivity can be made different depending on the positions in the longitudinal direction of the sensor. By mounting this sensor on the object to be detected, when the object to be detected has a position for intensively detecting the presence of the liquid and a position for preliminarily detecting the presence of the liquid, such as a diaper, the presence of the liquid can be detected depending on the positions. By providing the communication device which outputs the sensing result of the sensor by wireless communication and the receiver which receives the sensing result, the sensing result can be obtained from the signal received by the receiver. Therefore, it is possible to obtain the sensing result remotely, or obtain the sensing result without touching the object to be detected.

(12) The detection method according to an embodiment is a method for detecting the presence of a liquid, in which a first electrode and a second electrode each having a thread-like or band-like structure are arranged side by side in a direction intersecting a longitudinal direction; the first electrode and the second electrode are made of different materials, and generated power is generated between electrodes in response to the liquid being present between the electrodes; at least one of the first electrode and the second electrode includes a first portion having a first surface area at a first position in a longitudinal direction, and includes a second portion having a second surface area larger than the first surface area at a second position in the longitudinal direction different from the first position; and the fact that a predetermined amount of the liquid is present is detected by detecting that an amount of the generated power exceeds a threshold value. The threshold value is a value larger than a maximum value of an amount of generated power of the first portion and smaller than a maximum value of an amount of generated power of the second portion.

By using the sensor in which the second surface area of the second portion is larger than the first surface area of the first portion, the presence of the liquid can be detected with different sensitivities in the longitudinal direction of the sensor. By mounting this sensor on the object to be detected, when the object to be detected has a position for intensively detecting the presence of the liquid and a position for preliminarily detecting the presence of the liquid, such as a diaper, the presence of the liquid in the second portion can be detected by comparing the threshold value and the amount of power generated in the second portion.

<2. Examples of the Sensor, the Detection System, and the Detection Method>

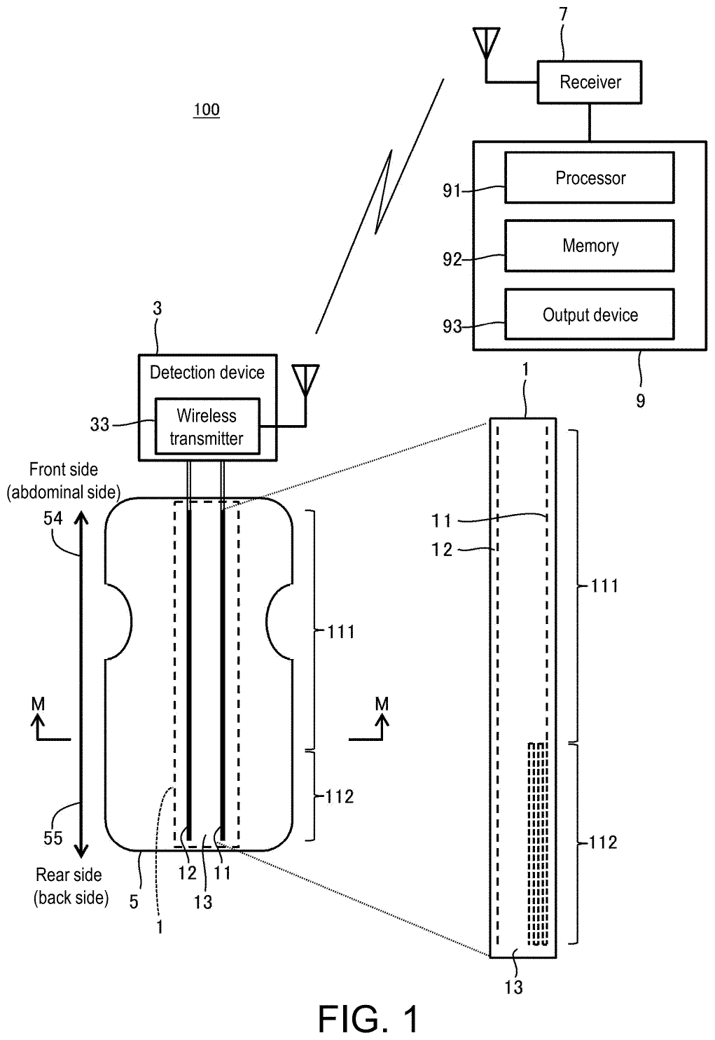

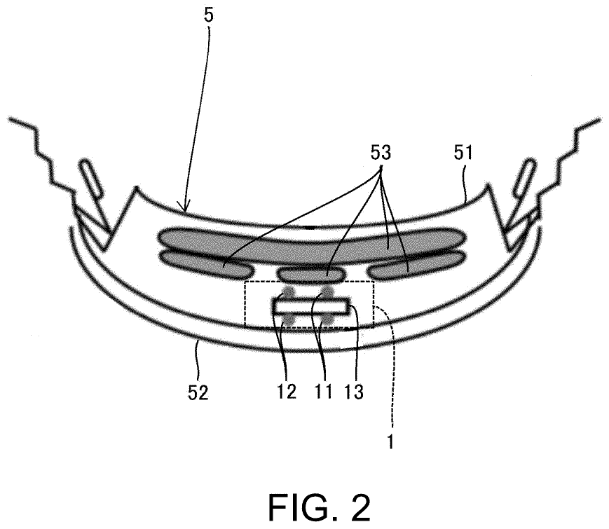

FIG. 1 is a schematic view of the detection system 100 according to the present embodiment. The detection system 100 is a system for detecting the presence of a liquid in an object to be detected. In the present embodiment, the object to be detected is an absorbing member body 5. The detection system 100 detects the liquid which exists in the absorbing member body 5 by a sensor 1 installed in the absorbing member body 5. FIG. 2 is a cross-sectional view of the absorbing member body 5 taken along the line M-M of FIG. 1.

In the present embodiment, the absorbing member body 5 has a basic configuration as a diaper. That is, the absorbing member body 5 includes a front sheet 51, a back sheet 52, and an absorbent 53. The absorbent 53 is arranged between the front sheet 51 and the back sheet 52. The absorbing member body 5 has a front side 54 located on the front side of a wearer and a rear side 55 located on the back side of the wearer when worn on the wearer. The wearer's excrement is given to the absorbent 53 from the side of the front sheet 51. The absorbing member body 5 has a substantially rectangular shape in a plan view in a deployed state.

The front sheet 51 is a substantially rectangular liquid permeable sheet. The front sheet 51 is made of, for example, a non-woven fabric or a woven fabric. The front sheet 51 comes into contact with the wearer's skin when worn on the wearer. The front sheet 51 is configured to improve the permeability of the liquid so that the permeating liquid does not easily return to the wearer side. Thus, the urine excreted from the wearer can rapidly permeate into the absorbent 53. Thus, even if the wearer urinates, the front sheet 51 does not substantially retain urine and is in a substantially dry state as long as the absorbent 53 has a surplus capacity for water absorption. As a result, contact between urine and the wearer's skin is suppressed.

The back sheet 52 is a substantially rectangular non-liquid permeable sheet. The back sheet 52 is made of a waterproof material having a waterproof film or the like. The back sheet 52 prevents the urine absorbed by the absorbent 53 from leaking to the outside.

The absorbent 53 is composed of an absorbent fiber such as pulp and a highly absorbent polymer. The highly absorbent polymer allows many liquids to be retained in the absorbent 53. The absorbent 53 is a substantially rectangular mat body which is long in the front-rear direction. The absorbent 53 is arranged across the front side 54 and the rear side 55 of the absorbing member body 5 with the longitudinal direction substantially coinciding with the front-rear direction of the absorbing member body 5. Therefore, the absorbent 53 can absorb the urine excreted on the front side 54 and absorb the urine exceeding the absorption amount of the front side 54 on the rear side 55.

The sensor 1 is arranged between the absorbent 53 and the back sheet 52. That is, the sensor 1 is arranged on the side of the back sheet 52 of the absorbent 53. The sensor 1 functions as a urine power generation battery. The sensor 1 has a pair of electrodes 11 and 12. The electrodes 11 and 12 each have a thread-like or band-like structure. The sensor 1 is arranged so that the pair of electrodes 11 and 12 are in contact with the absorbent 53 at an interval in the width direction intersecting the longitudinal direction. The pair of electrodes 11 and 12 are arranged so as to extend in the front-rear direction along the absorbent 53 which is long in the front-rear direction. That is, the electrodes 11 and 12 are arranged with the longitudinal direction substantially coinciding with the longitudinal direction of the absorbent 53.

The electrodes 11 and 12 function as a positive electrode and a negative electrode, respectively. The electrodes 11 and 12 generate power by coming into contact with the liquid existing between the electrodes. The sensor 1 detects that the liquid is present between the electrodes 11 and 12 by the generated power of the electrodes 11 and 12.

At least one of the electrodes 11 and 12 is sewn on a fabric 13. In this example, both the electrodes 11 and 12 have a conductive thread-like structure and are sewn to the fabric 13. The electrode 11 which functions as the positive electrode is made of a silver thread as an example. The electrode 12 which functions as the negative electrode is made of an aluminum thread as an example. The fabric 13 is cloth and, for example, is made of cloth of a cotton material. The fabric 13 is arranged in contact with the absorbent 53 on the side of the back sheet 52. Thus, the electrodes 11 and 12 made of the threads sewn on the fabric 13 come into contact with the absorbent 53 on the side of the back sheet 52.

As at least one of the electrodes 11 and 12, the electrode 11 includes a first portion 111 having a first surface area S1 at a position (first position in the longitudinal direction) corresponding to the front side 54 of the absorbing member body 5, and has a second portion 112 having a second surface area S2 at a position (second position in the longitudinal direction) corresponding to the rear side 55 of the absorbing member body 5. The second surface area S2 is larger than the first surface area S1 (S2>S1). The surface area refers to the area in contact with the absorbent 53, and refers to the surface area of the portion of the electrodes 11 and 12 made of threads sewn on the fabric 13 exposed to at least the surface of the fabric 13 on the side of the absorbent 53.

The first portion 111 and the second portion 112 of the electrode 11 have different thread sewing structures for the fabric 13. The sewing structure refers to a structure which the thread forms with respect to the fabric as the thread is sewn on the fabric 13. The difference in the sewing structure between the first portion 111 and the second portion 112 is, for example, that the sewing method of the thread forming the electrode 11 is different between the first portion 111 and the second portion 112. Specifically, the second portion 112 is sewn by a sewing method having a higher density with respect to the fabric 13 per unit than the first portion 111.

As an example, the sewing method having a high density with respect to the fabric 13 per unit is a sewing method in which a large area of the thread forming the electrode 11 appears on the surface of the fabric 13 on the side of the absorbent 53. For example, the first portion 111 is sewn to the fabric 13 by wave stitching, and the second portion 112 is sewn to the fabric 13 by back stitching. The back stitching refers to lock stitching or half back stitching.

As another example, as illustrated in FIG. 1, the second portion 112 is sewn with a sewing length longer than the first portion 111. In the example of FIG. 1, the first portion 111 is sewn once by wave stitching, whereas the second portion 112 is sewn by a sewing method having an overlap with respect to the sewing direction of the thread.

The fabric 13 is made of cloth, and may function as a part of the absorbent 53. In this case, the electrodes 11 and 12 sewn on the fabric 13 are arranged with a water absorbent in between.

The sewing method having a high density with respect to the fabric 13 per unit in this case is, for example, a sewing method in which the thread forming the electrode 11 is sewn to the fabric 13 at a short pitch. Specifically, the first portion 111 is sewn to the fabric 13 by wave stitching with a long pitch, and the second portion 112 is sewn to the fabric 13 by wave stitching with a short pitch.

Thus, the thread forming the electrode 11 is exposed to the surface of the fabric 13 on the side of the absorbent 53 more in the second portion 112 than in the first portion 111. Therefore, the electrode 11 is in contact with the absorbent 53 more in the second portion 112 than in the first portion 111.

The above example illustrates a case where both the electrodes 11 and 12 have a conductive thread-like structure, but one of the electrodes 11 and 12 may have a conductive band-like structure.

Since the electrodes 11 and 12 are arranged in contact with the absorbent 53, the absorbent 53 which has absorbed the liquid serves as a current generation path between the electrodes 11 and 12. Therefore, a current is generated at the electrodes 11 and 12 as the electrodes 11 and 12 are in contact with the liquid absorbed by the absorbent 53. Since the electrodes 11 and 12 generate power by the liquid absorbed by the absorbent 53, the sensor 1 is used for detecting the liquid absorption to the absorbent 53.

The sensor 1 is connected to a detection device 3. The detection device 3 is connected to the ends of the electrodes 11 and 12 on the front side 54. Thus, the detection device 3 is prevented from pressing the body when the wearer wearing the absorbing member body 5 is in a supine position.

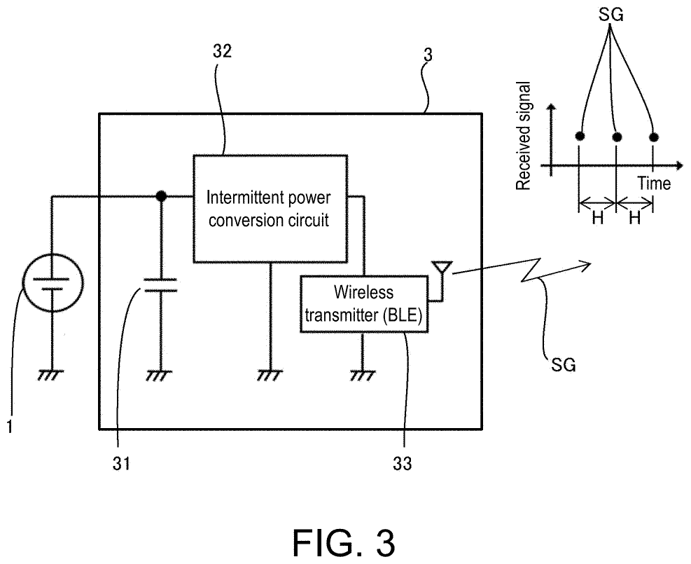

FIG. 3 is a schematic view illustrating the configuration of the detection device 3. With reference to FIG. 3, the detection device 3 includes a capacitor 31. The capacitor 31 is connected to the sensor 1 and stores the power generated by the electrodes 11 and 12.

The detection device 3 includes an intermittent power conversion circuit 32. The capacitor 31 is connected to the power supply terminal of the intermittent power conversion circuit 32. The intermittent power conversion circuit 32 operates using the capacitor 31 as an operating power supply. The intermittent power conversion circuit 32 monitors the charge voltage of the capacitor 31.

A wireless transmitter 33 is connected to the intermittent power conversion circuit 32. The wireless transmitter 33 performs wireless communication with a receiver 7. The wireless communication is, for example, Bluetooth®, Bluetooth Low Energy® or the like.

When the intermittent power conversion circuit 32 detects that the charge voltage of the capacitor 31 reaches a set voltage Vt set as the driving condition of the wireless transmitter 33, the intermittent power conversion circuit 32 supplies the power charged in the capacitor 31 to the wireless transmitter 33. Thus, a radio signal SG is output from the wireless transmitter 33.

When the power of the capacitor 31 is consumed by supplying power from the intermittent power conversion circuit 32 to the wireless transmitter 33, the potential of the capacitor 31 drops, and the intermittent power conversion circuit 32 stops operating. Thus, the supply of power to the wireless transmitter 33 is stopped. If the electrodes 11 and 12 are generating power, the capacitor 31 is charged again.

If the electrodes 11 and 12 are generating power, the capacitor 31 repeats charging and discharging. Along with this, the output of the radio signal SG from the wireless transmitter 33 becomes intermittent. Therefore, the radio signal SG output from the wireless transmitter 33 is a detection signal indicating the detection of the liquid existing between the electrodes 11 and 12.

The interval H of the output of the radio signal SG from the wireless transmitter 33 depends on the charging speed to the capacitor 31. The charging speed increases as the amount of power generation increases. Therefore, the interval H of the output of the radio signal SG from the wireless transmitter 33 becomes shorter as the amount of power generation of the electrodes 11 and 12 increases, and becomes longer as the amount of power generation decreases.

The amount of power generation increases as there is more contact between the electrodes 11 and 12 and the liquid. That is, since the electrodes 11 and 12 are arranged in contact with the absorbent 53 with the longitudinal direction substantially coinciding with the longitudinal direction of the absorbent 53, the amount of power generation increases as the amount of liquid absorbed by the absorbent 53 increases. Therefore, the interval H of the output of the radio signal SG from the wireless transmitter 33 represents the amount of liquid absorbed by the absorbent 53.

The radio signal SG transmitted from a transmitter 95 is received by the receiver 7. The receiver 7 is connected to a management device 9 and gives the received radio signal SG to the management device 9. The management device 9 is a computer having a processor 91 and a memory 92, and is, for example, a terminal device such as a smartphone.

The processor 91 can perform processing related to the degree of water absorption of the absorbent 53 of the absorbing member body 5 by executing a program stored in the memory 92. The processing related to the degree of water absorption of the absorbent 53 of the absorbing member body 5 is, for example, processing of determining the necessity of replacing the diaper. When the liquid reaches the rear side 55 of the absorbent 53, it is taken as the replacement time of the absorbing member body 5. When it is determined necessary to replace the diaper, an output device 93 of the management device 9 outputs that it is necessary to replace the diaper.

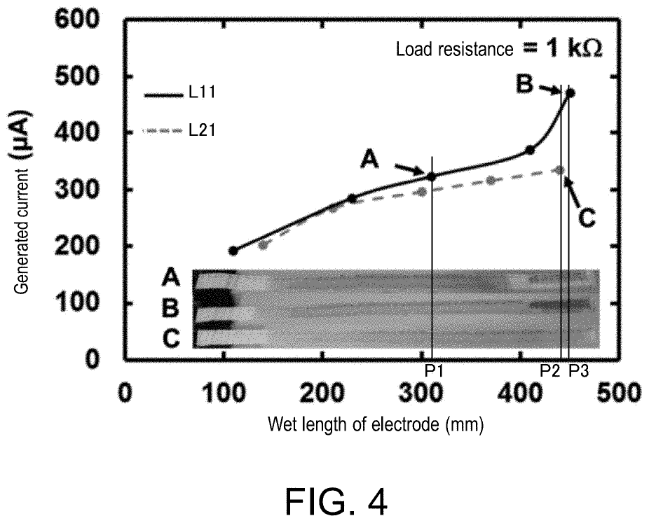

The inventors arranged the sensor 1 having the electrode 11 made of a silver thread and the electrode 12 made of an aluminum thread on the absorbing member body 5, and measured the generated current of the sensor 1 while injecting a liquid into the absorbing member body 5 to obtain and the generated current characteristic of FIG. 4. In the measurement, the liquid was injected from the front side 54 of the absorbing member body 5, and the generated current of the sensor 1 was measured with the passage of time. The passage of time is represented in FIG. 4 by the length of the absorbent 53 absorbing the liquid in the longitudinal direction from the end of the front side 54, that is, the length of the electrode in contact with the liquid (wet length). The horizontal axis of FIG. 4 represents the wet length, and the vertical axis represents the amount of the generated current.

The measurement result L11 of FIG. 4 represents the measurement result of the generated current in the sensor 1 in which the electrode 12 was formed by wave stitching an aluminum thread to the fabric 13 of a cotton material, and the electrode 11 was formed by overlapping wave stitching in the first portion 111 and lock stitching in the second portion 112 five times in the sewing direction. The measurement result L21 of FIG. 4 is the measurement result as a comparative example, and represents the result obtained by measuring the generated current, under the same conditions as the sensor 1, in a sensor in which the electrode 11 was formed by wave stitching a silver thread without overlapping in the sewing direction. The length of the electrodes 11 and 12 of the sensor 1 used in the longitudinal direction was 520 mm, the first portion 111 of the electrode 11 was 440 mm, and the second portion 112 was 80 mm.

The value A represents the measured value of the measurement result L11 when the wet length was about 310 mm. The position P1 about 310 mm from the end of the front side 54 of the absorbing member body 5 is a point included in the first portion 111. Therefore, the value A is the measured value in a state where the liquid was absorbed by the absorbent 53 up to the position P1 and the liquid was not absorbed in the second portion 112.

The value B represents the measured value of the measurement result L11 when the wet length was about 450 mm. The value C represents the measured value of the measurement result L21 when the wet length was about 440 mm. The position P2 about 440 mm from the end of the front side 54 of the absorbing member body 5 and the position P3 about 450 mm from the end of the front side 54 of the absorbing member body 5 are points included in the second portion 112. Therefore, both the values B and C are the measured values in a state where the liquid was absorbed up to the second portion 112.

With reference to FIG. 4, it was confirmed in both the measurement results L11 and L21 that the generated current tends to increase as the wet length of the electrode becomes longer. The generated current characteristic was obtained that when the wet length becomes larger than 440 mm, which is the first portion 111, the rate of increase of the measured value in the measurement result L11 becomes sharply larger than the rate of increase of the measured value in the measurement result L21, and the value B is 150 μA or more larger than the value C.

From the generated current characteristic of FIG. 4, it was verified that by setting the second surface area S2 of the second portion 112 of the electrode 11 larger than the first surface area S1 of the first portion 111, the amount of power generation when the liquid reaches the second portion 112 can be made much larger than the amount of power generation when the liquid is absorbed up to the first portion 111, compared to the case where the surface area is not changed. That is, it was verified that the amounts of power generation of the sensor 1 before and after the liquid reaches the second portion 112 of the absorbent 53 can be made significantly different.

In the sensor 1, the amount of power generation when the liquid reaches the second portion 112 is made much larger than the amount of power generation when the liquid is absorbed up to the first portion 111, which can increase the difference between the charging speeds to the capacitor 31 when the liquid is absorbed up to the first portion 111 and when the liquid reaches the second portion 112.

When detecting water supply to the second portion 112, it is conceivable to provide the electrode 11 or 12 in only the second portion 112, that is, only the rear side 55. However, by arranging the electrode 11 up to the first portion 111, the sensor 1 can generate power in the entire longitudinal direction, and increase the amount of power generation.

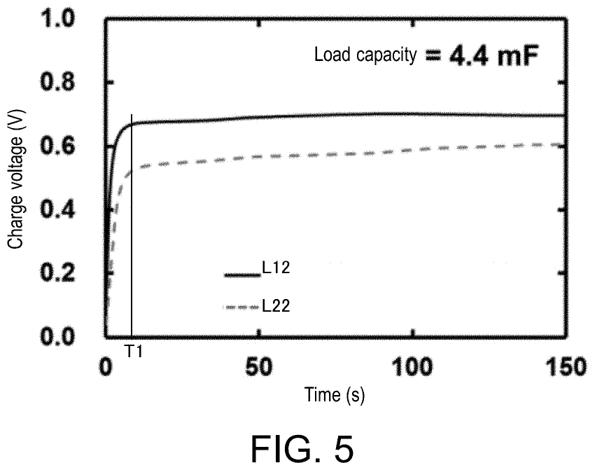

The inventors connected the detection device 3 to the sensor 1 used for the measurement of FIG. 4 and the sensor according to the comparative example, and measured the change over time of the charge voltage of the capacitor 31 to obtain the charging characteristic of FIG. 5. The generated power of the sensor 1 and the sensor according to the comparative example, in a state where the liquid was absorbed by the absorbent 53 up to the points P2 and P3 at which the values B and C in FIG. 4 were obtained, was used for charging the capacitor 31. An aluminum electrolytic capacitor having a load capacity of 4.4 mF was used as the capacitor 31. The horizontal axis of FIG. 5 represents the time elapsed from the start of charging, and the vertical axis represents the charge voltage.

The measurement result L12 of FIG. 5 represents the measurement result of the charge voltage when the sensor 1 was used, and the measurement result L22 represents the measurement result of the charge voltage when the sensor according to the comparative example was used. In both the measurement results L12 and L22, the charge voltage rose sharply at time T1 which was about 10 seconds after the start of charging, and then gradually rose.

In FIG. 5, in the measurement result L12, the charge voltage exceeded 0.6 V at time T1, whereas in the measurement result L22, the charge voltage at time T1 rose only to about 0.5 V. In the measurement result L22, it took 150 seconds from the start of charging for the charge voltage to reach 0.6 V due to the gradual rise after time T1.

From this result, it was found that by setting the second surface area S2 of the second portion 112 larger than the first surface area Si of the first portion 111, the capacitor 31 can be charged quickly, compared to the case where the second surface area S2 is not larger. In other words, it was verified that the charging speeds of the capacitor 31 before and after the liquid reaches the second portion 112 of the absorbent 53 can be made significantly different.

Based on these verifications, the set voltage Vt of the intermittent power conversion circuit 32 of the detection device 3 is set lower than the charge voltage of the capacitor 31 when the sensor 1 is used at time T1 and higher than the charge voltage when the sensor according to the comparative example is used. In other words, it is a value larger than the maximum value of the amount of power generation in the first portion 111 and smaller than the maximum value of the amount of power generation in the second portion 112. In the case of the sensor 1 used in the verifications of FIG. 4 and FIG. 5, the set voltage Vt is set to 0.6 V or more (Vt≤0.6 V) as an example. Preferably, the set voltage Vt is 0.6 V (Vt=0.6 V).

Thus, the wireless transmitter 33 is not activated in a state where the liquid is absorbed by the absorbent 53 to a position corresponding to the first portion 111 and does not reach a position corresponding to the second portion 112. As a result, the radio signal SG is not output in this state. When the liquid reaches the second portion 112, the wireless transmitter 33 is activated. As a result, the radio signal SG is output. Therefore, by receiving the radio signal SG at the receiver 7, that is, by making the surface areas different in the longitudinal direction in this way, the accuracy of detecting the liquid in the longitudinal direction of the sensor 1 can be made different. The management device 9 can determine that the liquid has been sufficiently absorbed by the absorbent 53, that is, the key to the effect of a diaper.

The inventors constructed the detection system 100 by mounting the sensor 1 used for the measurements of FIG. 4 and FIG. 5 on the absorbing member body 5 (diaper), and carried out an experiment of detecting the replacement time of the absorbing member body 5 with the detection system 100. The absorbing member body 5 used was injected with water from the front side 54, so that the water absorption of the absorbent 53 proceeded from the front side 54 to the rear side 55. The absorbent 53 was in a state of absorbing water up to the rear side 55 with injection of about 600 ml of water in total. The liquid used was physiological saline.

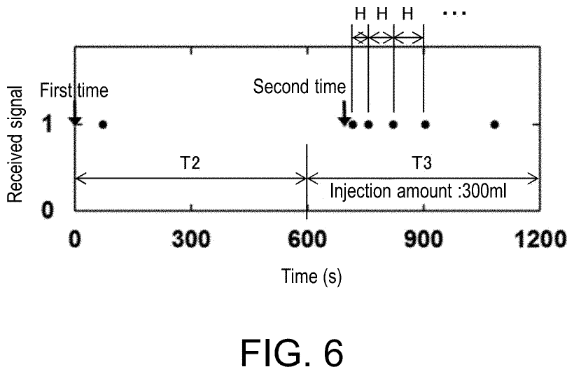

In the experiment, the amount of liquid injected was one injection, and the second injection was performed after 600 seconds or more had elapsed from the first injection, and a total of 600 ml of liquid was injected into the absorbing member body 5. Whether the radio signal SG was received by the receiver 7 from the start of injection was measured, and the measurement result of FIG. 6 was obtained. The horizontal axis of FIG. 6 represents the time elapsed from the start of injection, and the vertical axis represents whether the radio signal SG was received. The value “0” indicates no reception, and the value “1” indicates reception.

From the result of FIG. 6, the radio signal SG was received once in the period T2 after the first injection and before the second injection after 71 seconds. In the period T3 after the second injection, the radio signal SG was received once 22 seconds after the injection and then four times. During the period T3, the radio signal SG was received 5 times in 600 seconds.

From this result, it can be seen that the amount of power generation of the sensor 1 increased more rapidly in the period T3 than in the period T2. In other words, it can be seen that in the first injection, the first portion 111 was in contact with the liquid and the second portion 112 was not in contact with the liquid, whereas in the second injection, the second portion 112 was in contact with the liquid. Therefore, by using the detection system 100, it was verified that when the reception interval H of the radio signal SG is smaller than a threshold value and the radio signal SG is received more than a predetermined number of times, it is determined in the management device 9 that it is time to replace the diaper.

In the management device 9, the threshold value of the reception interval H of the radio signal SG is set to a value shorter than the reception interval at the maximum power generation amount of the first portion 111 and longer than the reception interval at the maximum power generation amount of the second portion 112. Thus, the threshold value of the amount of generated power can be set to a value larger than the maximum value of the amount of generated power of the first portion 111 and smaller than the maximum value of the amount of generated power of the second portion 112, and exceeding this threshold value can be detected by the reception interval being shorter than the threshold value of the reception interval. That is, this detection system 100 can reliably detect that the liquid has come into contact with the second portion 112, and can reliably determine the time to replace the diaper.

<3. Addendum> The present invention is not limited to the above embodiment, and various modifications are possible.

What is claimed is:

1. A sensor for detecting presence of a liquid, comprising:
a first electrode and a second electrode,
wherein the first electrode and the second electrode each have a thread-like or band-like structure, and are arranged side by side in a direction intersecting a longitudinal direction, and
at least one of the first electrode and the second electrode comprises a first portion having a first surface area at a first position in the longitudinal direction, and comprises a second portion having a second surface area larger than the first surface area at a second position in the longitudinal direction different from the first position.

2. The sensor according to claim 1, wherein the first electrode and the second electrode are made of different materials, and generated power is generated between electrodes in response to the liquid being present between the electrodes, and
second generated power generated by the second portion is larger than first generated power generated by the first portion.

3. The sensor according to claim 2, further comprising an output part which outputs a detection signal indicating detection of the liquid,
wherein the output part is connected to the first electrode and the second electrode.

4. The sensor according to claim 1, wherein the first electrode and the second electrode are provided so as to be in contact with an absorbent of a worn article having the absorbent which receives excrement, and
the first position is located in front of the second position in the case of a wearer wears the worn article.

5. The sensor according to claim 1, wherein at least one of the first electrode and the second electrode is sewn on a fabric.

6. The sensor according to claim 5, wherein the first portion and the second portion have different sewing structures of the electrodes with respect to the fabric.

7. The sensor according to claim 6, wherein the sewing structure of the second portion has a higher density of electrode with respect to the fabric per unit than the sewing structure of the first portion.

8. The sensor according to claim 7, wherein the first electrode and the second electrode are in contact with each other via a water absorbent,
at least one of the first electrode and the second electrode is sewn with the water absorbent as the fabric, and
the sewing structure of the second portion has a shorter sewing pitch than the sewing structure of the first portion.

9. The sensor according to claim 6, wherein the sewing structure of the second portion has a larger area of electrode appearing on a surface of the fabric in contact with the liquid than the sewing structure of the first portion.

10. The sensor according to claim 9, wherein the sewing structure of the second portion has an overlap with respect to a sewing direction of the electrode.

11. A detection system for detecting presence of a liquid in an object to be detected, comprising:
a sensor having a first electrode and a second electrode and mounted on the object to be detected;
a communication device connected to the sensor and outputting a sensing result of the sensor by wireless communication; and
a receiver receiving the sensing result,
wherein the first electrode and the second electrode each have a thread-like or band-like structure, and are arranged side by side in a direction intersecting a longitudinal direction, and
at least one of the first electrode and the second electrode comprises a first portion having a first surface area at a first position in the longitudinal direction, and comprises a second portion having a second surface area larger than the first surface area at a second position in the longitudinal direction different from the first position.

12. A detection method for detecting presence of a liquid, comprising:
arranging a first electrode and a second electrode each having a thread-like or band-like structure side by side in a direction intersecting a longitudinal direction;
the first electrode and the second electrode being made of different materials, and generated power being generated between electrodes in response to the liquid being present between the electrodes;
at least one of the first electrode and the second electrode comprising a first portion having a first surface area at a first position in a longitudinal direction, and comprising a second portion having a second surface area larger than the first surface area at a second position in the longitudinal direction different from the first position; and
detecting that a predetermined amount of the liquid is present by detecting that an amount of the generated power exceeds a threshold value,
wherein the threshold value is a value larger than a maximum value of an amount of generated power of the first portion and smaller than a maximum value of an amount of generated power of the second portion.
